SEQUENCE LISTING (37 sequences)

SEQ ID NO: 1 - DNA, 15 bp, Artificial Sequence
  Description of Artificial Sequence Synthetic oligonucleotide
ggaagcctagctgga

SEQ ID NO: 2 - DNA, 16 bp, Artificial Sequence
  Description of Artificial Sequence Synthetic oligonucleotide
ggaagcctagctggac

SEQ ID NO: 3 - DNA, 20 bp, Artificial Sequence
  Description of Artificial Sequence Synthetic oligonucleotide
aattctccagctaggcttcc

SEQ ID NO: 4 - DNA, 14 bp, Artificial Sequence
  Description of Artificial Sequence Synthetic oligonucleotide
ggtgagcactcatc

SEQ ID NO: 5 - DNA, 16 bp, Artificial Sequence
  Description of Artificial Sequence Synthetic oligonucleotide
gatgagtgctgacctt

SEQ ID NO: 6 - DNA, 16 bp, Artificial Sequence
  Description of Artificial Sequence Synthetic primer
ccttcggatcgacctg

SEQ ID NO: 7 - DNA, 16 bp, Artificial Sequence
  Description of Artificial Sequence Synthetic primer
ctactcacgagtggaa

SEQ ID NO: 8 - DNA, 16 bp, Artificial Sequence
  Description of Artificial Sequence Synthetic oligonucleotide
gacgatgagtcctgac

SEQ ID NO: 9 - DNA, 18 bp, Artificial Sequence
  Description of Artificial Sequence Synthetic oligonucleotide
cggtcaggactcatcgtc

SEQ ID NO: 10 - DNA, 18 bp, Artificial Sequence
  Description of Artificial Sequence Synthetic oligonucleotide
aattggtacgcagtctac

SEQ ID NO: 11 - DNA, 14 bp, Artificial Sequence
  Description of Artificial Sequence Synthetic oligonucleotide
gtagactgcgtacc

SEQ ID NO: 12 - DNA, 18 bp, Artificial Sequence
  Description of Artificial Sequence Synthetic primer
gacgatgagtcctgaccg

SEQ ID NO: 13 - DNA, 18 bp, Artificial Sequence
  Description of Artificial Sequence Synthetic primer
gtagactgcgtaccaatt

SEQ ID NO: 14 - DNA, 28 bp, Artificial Sequence
  Description of Artificial Sequence Synthetic oligonucleotide
  modified_base (22)..(28): a, c, g, t, unknown or other
ggaagcctagctggacaattcnnnnnnn

SEQ ID NO: 15 - DNA, 28 bp, Artificial Sequence
  Description of Artificial Sequence Synthetic oligonucleotide
  modified_base (26)..(28): a, c, g, t, unknown or other
ggaagcctagctggacaattccracnnn

SEQ ID NO: 16 - DNA, 29 bp, Artificial Sequence
  Description of Artificial Sequence Synthetic oligonucleotide
  modified_base (27)..(29): a, c, g, t, unknown or other
ggaagcctagctggacaattcttcacnnn

SEQ ID NO: 17 - DNA, 28 bp, Artificial Sequence
  Description of Artificial Sequence Synthetic oligonucleotide
  modified_base (26)..(28): a, c, g, t, unknown or other
ggaagcctagctggacaattcgactnnn

SEQ ID NO: 18 - DNA, 17 bp, Artificial Sequence
  Description of Artificial Sequence Synthetic oligonucleotide
  modified_base (1): a, c, g, t, unknown or other
naaggtgagcactcatc

SEQ ID NO: 19 - DNA, 20 bp, Artificial Sequence
  Description of Artificial Sequence Synthetic oligonucleotide
gatcctccagctaggcttcc

SEQ ID NO: 20 - DNA, 28 bp, Artificial Sequence
  Description of Artificial Sequence Synthetic oligonucleotide
  modified_base (22)..(28): a, c, g, t, unknown or other
ggaagcctagctggacgatccnnnnnnn

SEQ ID NO: 21 - DNA, 27 bp, Artificial Sequence
  Description of Artificial Sequence Synthetic oligonucleotide
  modified_base (25)..(27): a, c, g, t, unknown or other
ggaagcctagctggacgatccracnnn

SEQ ID NO: 22 - DNA, 27 bp, Artificial Sequence
  Description of Artificial Sequence Synthetic oligonucleotide
  modified_base (25)..(27): a, c, g, t, unknown or other
ggaagcctagctggacgatcctcannn

SEQ ID NO: 23 - DNA, 27 bp, Artificial Sequence
  Description of Artificial Sequence Synthetic oligonucleotide
  modified_base (25)..(27): a, c, g, t, unknown or other
ggaagcctagctggacgatcccacnnn

SEQ ID NO: 24 - DNA, 28 bp, Artificial Sequence
  Description of Artificial Sequence Synthetic oligonucleotide
  modified_base (26)..(28): a, c, g, t, unknown or other
ggaagcctagctggacgatcctactnnn

SEQ ID NO: 25 - DNA, 27 bp, Artificial Sequence
  Description of Artificial Sequence Synthetic oligonucleotide
  modified_base (25)..(27): a, c, g, t, unknown or other
ggaagcctagctggacaatccracnnn

SEQ ID NO: 26 - DNA, 16 bp, Artificial Sequence
  Description of Artificial Sequence Synthetic oligonucleotide
gtccagctaggcttcc

SEQ ID NO: 27 - DNA, 27 bp, Artificial Sequence
  Description of Artificial Sequence Synthetic oligonucleotide
  modified_base (21)..(27): a, c, g, t, unknown or other
ggaagcctagctggaggcggnnnnnnn

SEQ ID NO: 28 - DNA, 27 bp, Artificial Sequence
  Description of Artificial Sequence Synthetic oligonucleotide
  modified_base (22)..(27): a, c, g, t, unknown or other
ggaagcctagctggaggcggannnnnn

SEQ ID NO: 29 - DNA, 27 bp, Artificial Sequence
  Description of Artificial Sequence Synthetic oligonucleotide
  modified_base (22)..(27): a, c, g, t, unknown or other
ggaagcctagctggaggcgggnnnnnn

SEQ ID NO: 30 - DNA, 27 bp, Artificial Sequence
  Description of Artificial Sequence Synthetic oligonucleotide
  modified_base (22)..(27): a, c, g, t, unknown or other
ggaagcctagctggaggcggtnnnnnn

SEQ ID NO: 31 - DNA, 14 bp, Artificial Sequence
  Description of Artificial Sequence Synthetic oligonucleotide
ggtgagcactcatc

SEQ ID NO: 32 - DNA, 16 bp, Artificial Sequence
  Description of Artificial Sequence Synthetic oligonucleotide
gatgagtgctgacctt

SEQ ID NO: 33 - DNA, 16 bp, Artificial Sequence
  Description of Artificial Sequence Synthetic oligonucleotide
  modified_base (7)..(16): a, c, g, t, unknown or other
gaggagnnnnnnnnnn

SEQ ID NO: 34 - DNA, 15 bp, Artificial Sequence
  Description of Artificial Sequence Synthetic oligonucleotide
  modified_base (6)..(15): a, c, g, t, unknown or other
gggacnnnnnnnnnn

SEQ ID NO: 35 - DNA, 17 bp, Artificial Sequence
  Description of Artificial Sequence Synthetic oligonucleotide
  modified_base (7)..(17): a, c, g, t, unknown or other
ggcggannnnnnnnnnn

SEQ ID NO: 36 - DNA, 14 bp, Artificial Sequence
  Description of Artificial Sequence Synthetic oligonucleotide
  modified_base (6)..(14): a, c, g, t, unknown or other
ggatgnnnnnnnnn

SEQ ID NO: 37 - DNA, 18 bp, Artificial Sequence
  Description of Artificial Sequence Synthetic oligonucleotide
ggaagcctagctggaggc

(19) United States
(12) Patent Application Publication
(10) Pub. No.: US 2007/0054272 A1
(43) Pub. Date: Mar. 8, 2007

(54) Method of preparing dna fragments by selective fragmentation of nucleic acids and applications thereof

(75) Inventors: Anne-Gaelle Brachet, Nantoin (FR); Philippe Rizo, La Tronche (FR); Pierre Taberlet, La Terrasse (FR); Isabelle Texier-Nogues, Grenoble (FR)

Correspondence Address: LOWE HAUPTMAN BERNER, LLP, 1700 DIAGONAL ROAD, SUITE 300, ALEXANDRIA, VA, 22314, US

(73) Assignees: Commissariat A L'ENERGIE ATOMIQUE, Paris (FR); UNIVERSITE JOSEPH FOURIER, Grenoble Cedex 9 (FR); CENTRE NATIONAL DE LA RECHERCHE SCIENTIFIQUE, Paris (FR)

(21) Appl. No.: 10/553,603

(22) PCT Filed: Apr. 19, 2004
(86) PCT No.: PCT/FR04/00963

§ 371 Date: Sep. 29, 2006

(30) Foreign Application Priority Data
Apr. 18, 2003 (FR) 0304893

(51) Int. Cl. C12Q 1/68; C07H 21/04

(52) U.S. Cl. 435/6; 536/24.3

(57) ABSTRACT

The invention relates to a method of preparing DNA fragments by selective fragmentation of nucleic acid fragments. The inventive method comprises a first selection step involving selection of short fragments, consisting in: a) preparing first double-stranded DNA fragments F1 using at least one restriction enzyme E1 which can randomly fragment the nucleic acid sample to be analysed, by generating said DNA fragments F1 with blunt or cohesive ends; b) ligating the ends of the DNA fragments F1 obtained in step (a) to at least one adapter AA′; c) cleaving the DNA fragments F1 obtained in step (b) using a restriction enzyme E2, such as to select a fraction of short fragments F2; and d) using any suitable means to purify the aforementioned fraction of short fragments F2. The inventive method also comprises the following optional step involving the second selection of one or more fragment sub-groups from the fraction of short fragments F2 obtained in step (d), said optional step consisting in: e) ligating the free end of the short fragments F2 obtained in step d) to at least one second complementary adapter BB′ (production of fragments F2); and f) amplifying short fragments F2. The invention also relates to the applications of the above-mentioned method for the analysis of genomes and transcriptomes.

The invention relates to a method of preparing DNA fragments by selective fragmentation of nucleic acids and to applications thereof for the analysis of genomes and transcriptomes.

Techniques for analyzing the genomes and transcriptomes of different species (animals, plants, microorganisms) or else of different subgroups or individuals within these species are based on the detection of one or more genetic marker(s) or footprint(s) by fragmentation of DNA (genome or cDNA) using one or more restriction enzymes, and then analysis, by any appropriate means, of the DNA fragments thus obtained.

These techniques have applications in extremely varied fields in biology, such as genetic mapping, the genotyping of species, of varieties, of individuals (animals, plants, microorganisms), the detection of polymorphism(s) (SNP or Single Nucleotide Polymorphism) in genes, associated with phenotypic characteristics, in particular with diseases, and also the establishment of gene expression profiles.

However, due in particular to the complexity of genomes, the techniques proposed do not allow a systematic high-throughput analysis of genomes and transcriptomes by automated techniques of the DNA chip hybridization type. Specifically:
the RFLP (Restriction Fragment Length Polymorphism) technique, which comprises analysis by Southern blotting of the fragments generated by restriction enzymes, has poor resolution insofar as it makes it possible to analyze only one or, at most, a few loci in a single reaction. In addition, the fragments obtained cannot be analyzed by DNA chip hybridization because the number of fragments generated is too great, resulting in saturation of the chip;
the AFLP (Amplified Fragment Length Polymorphism) technique described in European patent application (EP 0 534 858) in the name of Keygene, which has been adapted to DNA chip analysis (Jaccoud et al., N.A.R., 2001, 29, 4^{e}25), makes it possible to reduce the complexity of the starting sample to about a hundred fragments by selective amplification of a fraction of the fragments, by PCR using primers comprising, 3′ of the restriction site sequence, a specific sequence of a few bases (approximately 1 to 10). Thus, a pair of primers having n selective bases makes it possible, in theory, to amplify only a 1/4²ⁿ fraction of the fragments corresponding to those that have a sequence complementary to the selective sequence, i.e. 1/16th and 1/256th of the fragments for, respectively, n=1 and n=2;
the ligation-mediated selective PCR amplification technique described in particular in application EP 0 735 144 in the name of Research Development Corporation of Japan and the articles in the names of Zheleznaya et al., Biochemistry, 1995, 60, 1037-1043, and Smith et al., PCR Methods and Applications, 1992, 2, 21-27, makes it possible to reduce the complexity of the starting sample by selective amplification of a fraction of restriction fragments obtained by cleavage with a type IIS and, optionally, type IIN, and then ligation with one of the adapters complementary to the cohesive end generated by said type IIS and, optionally, IIN enzyme.

However, despite the reduction in complexity of the starting sample proposed in the above techniques, the hybridization of the targets obtained (PCR products of several hundred base pairs) on supports of the DNA chip type, i.e. of targets with probes of 10 to 20 bases, is often of poor quality (weak signals, false negatives and false positives) for the following reasons:
the presence of secondary structures in the target decreases the efficiency of hybridization of the probe due to the decrease in accessibility to the target and to the impossibility of optimizing the hybridization conditions because of the presence of a large number of fragments, that have different structures, to be hybridized with the same probe, and
nonspecific hybridization or crosshybridization reactions with “non-target” sequence having similarities with the target sequences result in false positives that reduce the ability of these techniques to detect small amounts of specific sequences and their ability to discriminate due to the increase in background noise.
U.S. Pat. No. 6,258,539 in the name of The Perkin-Elmer Corporation recommend hybridizing small targets (approximately 30 base pairs) on supports of the DNA chip type; the targets are generated from a restriction fragment representative of the cDNA to be analyzed, by: (i) ligation of one of the ends of the fragment with an adapter containing the recognition site for a type IIS restriction enzyme, and then cleavage of the 5′ end of said fragment with said type IIS enzyme. This technique is not suitable for the analysis of complex populations of nucleic acids such as genomes or transcriptomes, for which it is impossible to obtain a single restriction fragment representative of each molecule of interest to be analyzed.

It emerges from the above, that there is a real need for the provision of methods of analyzing genomes and transcriptomes that are more suited to practical needs, in particular in that they are at the same time reliable, reproducible, sensitive, specific, rapid and simple to carry out. Such methods, that thus make it possible to simultaneously analyze a large number of samples on supports of the DNA chip type, would therefore be completely suitable for the systematic analysis of genomes and transcriptomes for the abovementioned applications.

This is the reason for which the inventors have developed a method of preparing DNA fragments by selective fragmentation of nucleic acids (genomic DNA, cDNA reverse transcribed from mRNA), which advantageously makes it possible to obtain one or more set(s) of short DNA fragments (less than 100 bases or 100 base pairs) representative of the entire genome or transcriptome to be analyzed; this method thus makes it possible to obtain a hybridization that is at the same time rapid, efficient, reliable, reproducible, sensitive and specific for target nucleic acid molecules (DNA, RNA), with oligonucleotide probes immobilized on miniaturized supports of the DNA chip type; said method is useful both for the preparation of target DNAs capable of hybridizing with nucleotide probes, and in particular with oligonucleotide probes immobilized on miniaturized supports of the DNA chip type (detection of genetic marker(s) or footprint(s)) and for the preparation of DNA probes, in particular of DNA chips, capable of hybridizing with target nucleic acids (DNA, RNA) (preparation of genetic marker(s) or footprint(s)).

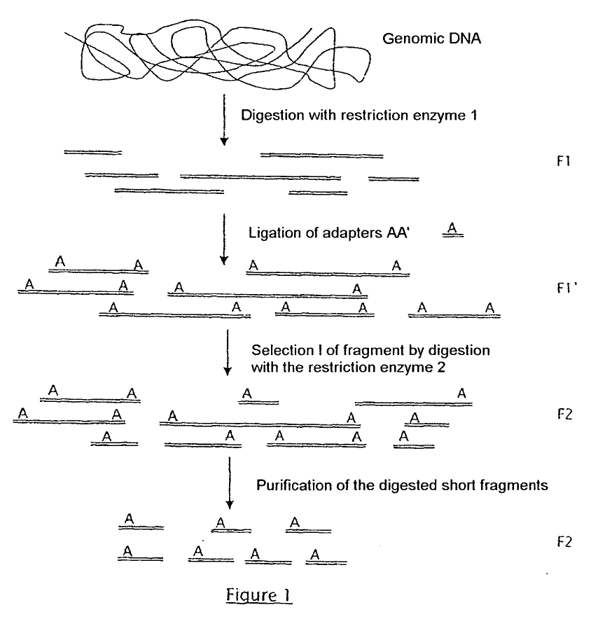

A subject of the present invention is thus a method of preparing DNA fragments from a sample of nucleic acids to be analyzed, which method is characterized in that it comprises the selective fragmentation of said nucleic acids by means of at least the following steps (FIG. 1):

I. a first selection of short fragments, comprising:
a) the preparation of first double-stranded DNA fragments F1 using at least one restriction enzyme E1 capable of randomly fragmenting the sample of nucleic acids to be analyzed, generating said DNA fragments F1 with blunt or cohesive ends,
b) the ligation of the ends of said DNA fragments F1 obtained in step a) to at least one adapter AA′, so as to form a unit—located at the junction of the complementary end of said adapter and of the 5′ end of said fragments F1, such that:
   the sequence of said unit is that of the first N-x base pairs of the recognition site—comprising N base pairs—of a restriction enzyme E2, the cleavage site of which is located downstream of said recognition site, with 1≦x≦N−1, and
   its 3′ end—located 5′ of said DNA fragments F1—is that of the restriction site of the restriction enzyme E1 (production of fragments F′1),
c) the cleavage of the DNA fragments F′1 obtained in b)—in the vicinity of their 5′ end—using said restriction enzyme E2, so as to select a fraction of short fragments F2,
d) the purification, by any appropriate means, of said fraction of short fragments F2, and, optionally,

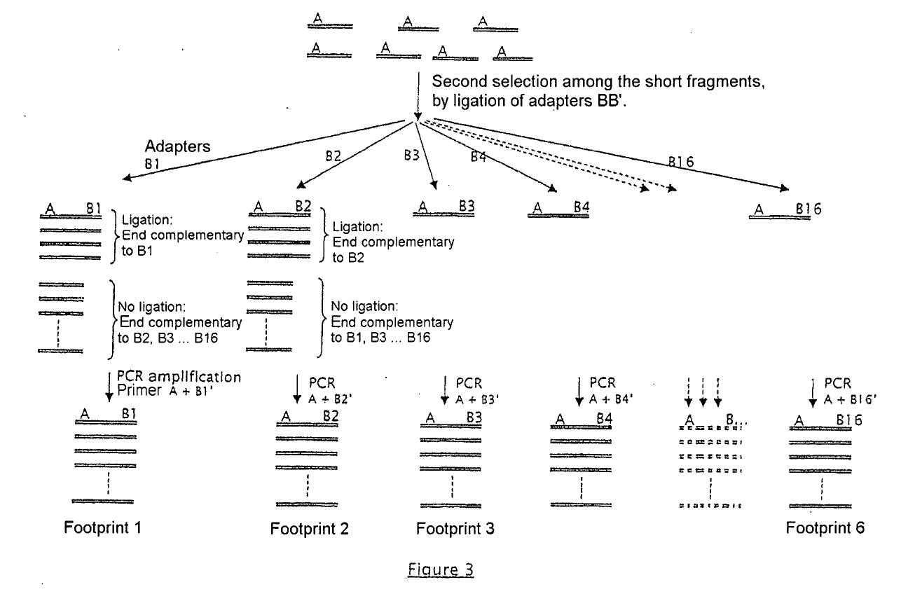

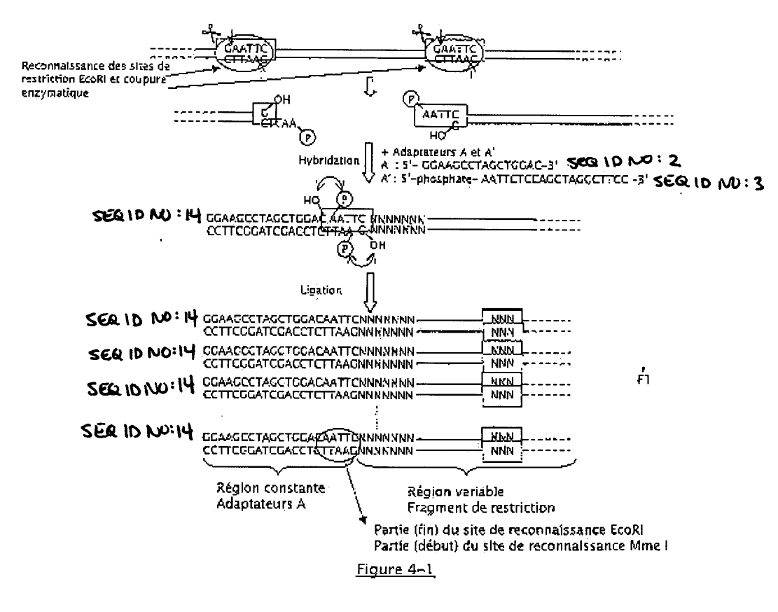

II. a second selection of one or more subset(s) of fragments from the fraction of short fragments F2 obtained in step d), in accordance with the following steps (FIG. 3):
e) the ligation of the free end (not linked to the adapter AA′) of short fragments F2 obtained in d) to at least a second complementary adapter BB′ (production of fragments F′2), and
f) the amplification of the short fragments F′2 linked to said adapters (AA′ and BB′), using at least one pair of appropriate primers, at least one being optionally labeled at its 5′ end, so as to select at least one subset of short fragments F′2 from the fraction of short fragments F2 obtained in d).

For the purpose of the present invention:
the term DNA fragment is intended to mean a double-stranded DNA fragment,
the term short fragment F2 or F′2 is intended to mean a DNA fragment of less than 100 base pairs,
the term long fragment F1 or F′1 is intended to mean a DNA fragment of several hundred base pairs,
the term adapter is intended to mean a double-stranded oligonucleotide of at least 6 base pairs,
the terms 5′ end and 3′ end, relating to a DNA fragment, an adapter or a site for recognition or cleavage by a restriction enzyme, are intended to mean, respectively, the 5′ end and the 3′ end of the positive strand of said DNA fragment, of said adapter or of said site for recognition or cleavage by a restriction enzyme,
the term free end, relating to a DNA fragment, is intended to mean the end that is not linked to an adapter,
the term complementary end of an adapter is intended to mean the end of said adapter that binds to the 3′ or 5′ end of a DNA fragment; when said adapter binds to the 5′ end of said DNA fragment, this involves the 3′ end of said adapter, and vice versa,
the term fraction of fragments is intended to mean a fraction of short fragments F2 prepared from the (long) fragments F1 obtained in step a) or a fraction of short fragments F′2 prepared from the short fragments F2; the terms “fraction”, “set” or “group” are considered to be equivalent and are used without any implied distinction in the subsequent text, and the same is true for the terms “subset(s)” and “subgroup(s)”,
the term cleavage site is intended to mean the restriction site of an endonuclease (restriction enzyme); in the subsequent text, the term “cleavage site” or “restriction site” is used without any implied distinction.

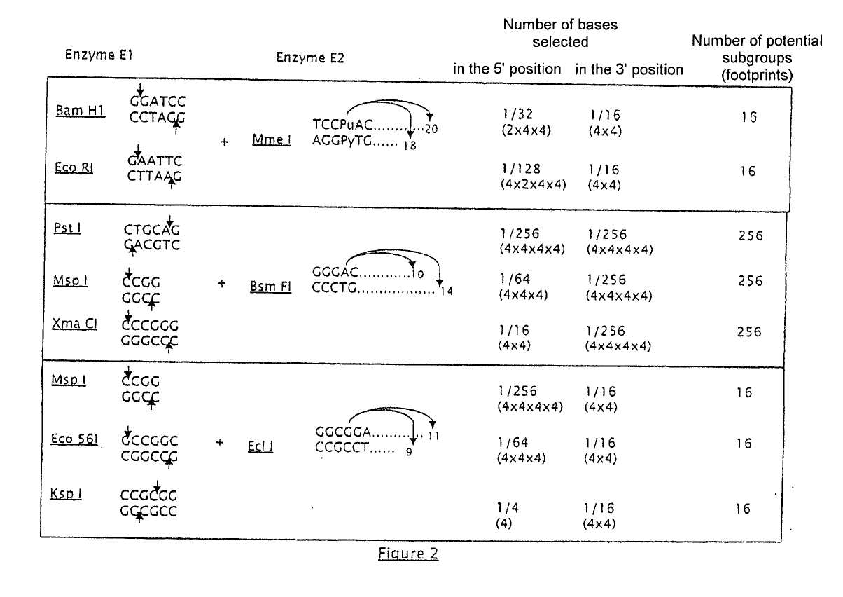

The combination of steps a) to d) of the method of preparing DNA fragments according to the invention advantageously makes it possible to, at the same time:
obtain short fragments F2 representative of the entire genome or transcriptome to be analyzed, i.e. having a length equivalent to that of oligonucleotide probes (FIG. 1), and
reduce the complexity of the sample to be analyzed by means of a selection, using the adapters AA′, of a fraction of short fragments F2 representative of the genome or transciptome to be analyzed (FIG. 2); such a selection makes it possible to avoid the problems of saturation of the support of the DNA chip type used for the hybridization.

The combination of steps a) to f) of the method of preparing DNA fragments according to the invention advantageously makes it possible to, at the same time (FIG. 2):
obtain short fragments F′2 representative of the entire genome or transcriptome to be analyzed, i.e. having a length equivalent to that of oligonucleotide probes (FIGS. 1 and 3);
reduce the complexity of the sample to be analyzed by means of a first and then a second selection, using the adapters AA′ and BB′, of one or more subsets of short fragments F′2 representative of the genome or transcriptome to be analyzed (FIG. 2); such a selection makes it possible to avoid the problems of saturation of the support of the DNA chip type used for the hybridization, and
detect, using the set of short fragments F2, a maximum number of different genetic footprints or markers representative of the genome or transcriptome to be analyzed, by means of a second selection of subsets of this set of short fragments F2 obtained in step d), using different adapters (B₁,B₁′, B₂B₂′, etc.) (FIGS. 2 and 3).

The use of such short fragments F2 or F′2 as targets or probes in DNA chip hybridization techniques has the following advantages compared with the techniques for analyzing genomes or transcriptomes of the prior art:

Reliability and Reproducibility

The fraction of short fragments F2 that is selected only by ligation of adapters and cleavage with restriction enzymes is representative of the entire genome or transcriptome to be analyzed. These short fragments, firstly, are easier to amplify and, secondly, make it possible to work on partially degraded DNA. In addition, the reduction of the complexity of the sample to be analyzed, by means of a first selection, using the adapter AA′ , of a fraction of short fragments F2 representative of the genome or transcriptome to be analyzed, which makes it possible to avoid the problems of saturation of the support of the DNA chip type used for the hybridization, also contributes to increasing the reliability and the reproducibility of the analysis of genomes or transcriptomes.

Sensitivity and Specificity

The sensitivity and the specificity of the hybridization are increased due to:
the reduction in size of the fragments to be hybridized (targets or probes of less than 100 bases or base pairs instead of several hundred bases or base pairs in the techniques of the prior art); this reduction decreases the crosshybridization reactions and the false positives by eliminating the “non-target sequences”, and increases the hybridization signal by decreasing the secondary structures of the DNA,
the harmonization of the hybridization conditions (temperature) for fragments of homogeneous size,
the purity of the DNA (elimination of the enzyme, buffers and long DNA fragments that remain).

Simplicity

The fragmentation of the nucleic acids (target or probe) comprises steps that are simple to carry out (enzymatic digestion, ligation). In addition, the optimization of the length, of the structure and of the composition of the DNA (target or probe) makes it possible to obtain a hybridization of good quality (no false positives, little background noise, etc.) and therefore to minimize the number of controls required and, consequently, to reduce the complexity of the chip.

Rapidity

The hybridization time is considerably reduced and is less than 1 h (approximately 15 to 20 min), instead of 12 h to 18 h in the techniques of the prior art.

Relatively Low Cost

The reduction in complexity of the chip makes it possible to reduce the cost of the latter.

Because of these various advantages, the method of preparing DNA fragments by selective fragmentation of nucleic acids according to the invention is particularly suitable for:
the rapid analysis of a large number of samples of target nucleic acids (genomic DNA or cDNA obtained by reverse transciption of mRNA) on DNA chips, and
the preparation of probes of small and controlled size from RNA or from genomic DNA, in particular for the fabrication of DNA chips on which said probes representing genetic markers for genomes or for transcriptomes are immobilized.

In accordance with the method of the invention, the double-stranded DNA fragments F1 of step a) are obtained by conventional techniques known in themselves. For example, the genomic DNA extracted from the sample to be analyzed is randomly fragmented using one or more restriction enzymes E1 that generate fragments with blunt or cohesive ends, selected according to their frequency of cleavage of the DNA to be analyzed, so as to obtain fragments that are less than 1000 bp, of the order of 200 to 400 bp. RNA (mRNA, genomic RNA of a microorganism, etc.) is extracted from the sample to be analyzed, converted to double-stranded cDNA by reverse transcription, and then fragmented in a manner similar to the genomic DNA. Among the restriction endonucleases E1 that can be used to cleave mammalian DNA, mention may be made, without implied limitation, of: EcoR I, BamH I, Pst I, Msp I, XmaC I, Eco 561, Ksp I, Dra I, Ssp I, Sac I, BbvC I, Hind III, Sph I, Xba I and Apa I.

In accordance with the invention, the enzyme E1 generates either blunt ends or cohesive ends; it preferably generates cohesive ends that have the advantage of allowing ligation with a single adapter.

In accordance with the method of the invention, the adapter as defined in step b) is an oligonucleotide of at least 6 bp, made up of two complementary strands (A and A′); said adapter, in b), is linked to the ends of said DNA fragment F1 by any appropriate means, known in itself, in particular using a DNA ligase such as T4 ligase.

In accordance with the method of the invention, steps a) and b) are carried out successively or simultaneously.

In accordance with the method of the invention, the 3′ end of the cleavage site of the restriction enzyme E1 and the 5′ end of the recognition site of the restriction enzyme E2 overlap over at least one base pair (FIG. 2), which makes it possible to select a fraction of short fragments F2 by cleavage with the restriction enzyme E2; these short fragments F2 are derived from the fraction of long fragments F′1 obtained in step b), which comprises the entire recognition site of said restriction enzyme E2 (N base pairs). Among the restriction enzymes E2, mention may be made, without implied limitation, of: Bpm I, Bsg I and BpuE I, which cleave 16 nucleotides downstream of their recognition site, and Eci I, BsmF I, Fok I, Mme I and Mbo II, which cleave, respectively, 11, 10, 9, 20 and 8 nucleotides downstream of their recognition site. In accordance with the method of the invention, the overlapping of said sites may be perfect (no mismatching) or it may comprise at least one mismatch (see, for example, the base pair located in the second position of the Ksp I site (restriction enzyme E1), which is not complementary to the base pair in the first position of the Eci I site (restriction enzyme E2) (FIG. 2)); in this case, the sequence of the recognition site of the restriction enzyme E2 is restored by ligation with an adapter whose end is complementary to said recognition site of the restriction enzyme E2 (adapter comprising the sequence “GGC” at the 3′ end of the strand A in the abovementioned example).

The number 1 to N−1 of base pairs of the recognition site of the restriction enzyme E2, located in the region of the junction of the complementary 3′ end of said adapter AA′ and of the 5′ end of said DNA fragments F1, and the length of said recognition site of the restriction enzyme E2, determine the fraction of short fragments that can be selected from the set of the (long) fragments F1 generated in step a); for an E2 recognition site of N bp and an overlap of N bp, between E1 and E2, the fraction of fragments selected corresponds to 1/4^{(N-n)}, this value being increased by a multiple of 2 for any purine or pyrimidine base pair recognized without distinction by said restriction enzyme E2 (Mme I enzyme, FIG. 2). Thus, the greater the overlap, the greater the number of fragments contained in the fraction (low factor of selection or of reduction of the complexity of the sample), and vice versa (high factor of selection or of reduction of the complexity of the sample) (FIG. 2).

In accordance with the method of the invention, the cleavage, at the 5′ end, of the long fragments F′1 in step c) makes it possible to obtain short DNA fragments F2 representative of the genome or transcriptome to be analyzed, that may contain a genetic marker capable of being detected by hybridization with a specific nucleotide probe, in particular an oligonucleotide complementary to said genetic marker. Alternatively, said fragments are immobilized on a solid support of the DNA chip type and are used as a genetic footprint or marker for analyzing genomes or transcriptomes.

In accordance with the method of the invention, the purification of the short fragments F2—optionally single-stranded and/or linked to an appropriate label (biotin, digoxigenin, fluoresceine)—(step d), is carried out by any appropriate means known in itself, for example: exclusion chromatography, filtration, precipitation with mixtures of ethanol and ammonium or sodium acetate, binding to a functionalized support (magnetic beads, beads made of a nonmagnetic polymer or a gold surface, coupled in particular to streptavidin or to an anti-digoxigenin or anti-fluoresceine antibody).

According to an advantageous embodiment of the method according to the invention, step a) is carried out with two different E1 restriction enzymes, E1_{A} and E1_{C}, such that:
at least one generates cohesive ends, different from those optionally generated by the other restriction enzyme, and
the 3′ end of the E1_{1A} restriction site is that of the unit as defined in step b).

According to an advantageous arrangement of this embodiment, one of the enzymes cleaves frequently and the other rarely.

Preferably, the enzyme that cleaves frequently is the enzyme E1_{A}, which enzyme E1_{A} generates at least one end of a fragment F1 that binds to the adapter AA′ in step b). The enzyme E1_{A} is related to the enzyme E2 insofar as the 3′ end of the E1_{A} restriction site corresponds to the first N-x base pairs of the E2 recognition site. The enzyme E1_{C} generates at least one end of a fragment F1—identical to or different from that generated by the enzyme E1_{A}, which end binds, in step b), to a second adapter CC′ that is different from the adapter AA′. Preferably, the 3′ end of the E1_{C} restriction site is different from that of the first N-x base pairs of the recognition site of the E2 enzyme, as defined in step b), so as not to reconstitute the sequence of the first N-x bases or base pairs of the recognition site of the restriction enzyme E2, by ligation of said adapter CC′ to at least one of the ends of said DNA fragments obtained in a).

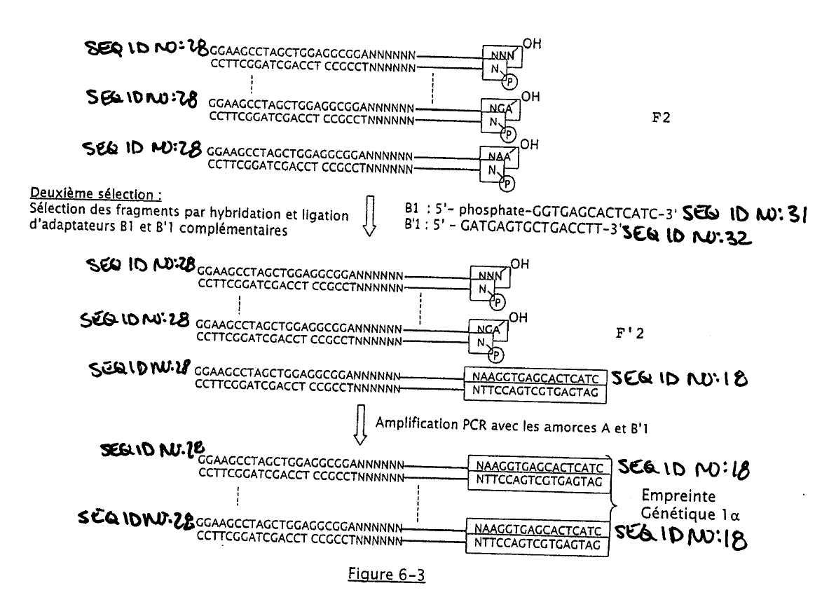

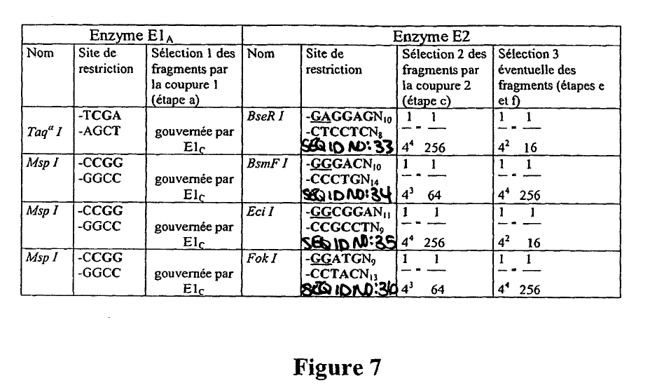

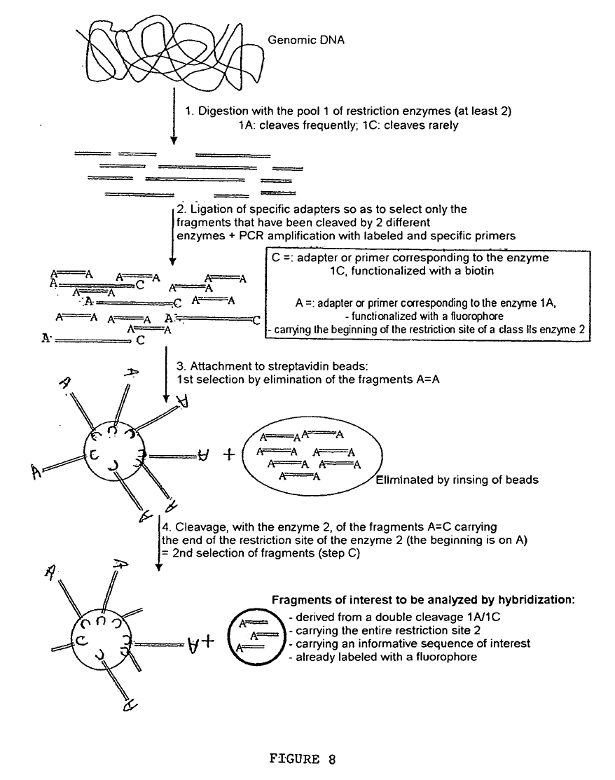

The use of such a pair of enzymes makes it possible to even further reduce the complexity of the sample to be analyzed by means of an additional selection of a set of fragments A=C, in particular by binding to a support functionalized with a ligand for the label linked to the 5′ end of the adapter CC′ (FIGS. 7 and 8).

By way of nonlimiting example of enzymes that cleave DNA frequently, mention may be made of those for which the restriction site has 4 base pairs, such as Msp I and Taq^{α} I.

By way of nonlimiting example of enzymes that cleave DNA rarely, mention may be made of those for which the restriction site has 5 or 6 base pairs, such as Pst I and EcoR I. According to an advantageous embodiment of the method according to the invention, it comprises an additional step consisting of the purification of the fragments less than 1000 bp, prior to the ligation step b). Said purification is carried out by any appropriate means known in itself, in particular by separation of the digestion products obtained in a) by agarose gel electrophoresis, visualization of the bands corresponding to the various fragments obtained, removal of the gel band or bands corresponding to the fragments less than 1000 bp and extraction of said double-stranded DNA fragments according to conventional techniques.

According to another advantageous embodiment of the method according to the invention, the adapter AA′ as defined in step b) comprises, at the 3′ end of the strand A and/or 5′ end of the strand A′, a zone 1 of approximately 1 to 8 bases or base pairs, which is partially or completely identical or complementary to the cleavage site of the enzyme E1 or E1_{A}, chosen so as to reconstitute the sequence of the first N-x bases or base pairs of the recognition site of the restriction enzyme E2, by ligation of said adapter AA′ to at least one of the ends of said DNA fragments obtained in a). Said zone 1 can optionally include one or more mismatches with the sequence of said cleavage site of the enzyme 1.

According to yet another advantageous embodiment of the method according to the invention, the adapter CC′ as defined above comprises, at the 3′ end of the strand C and/or 5′ end of the strand C′, a zone 1 of approximately 1 to 8 bases or base pairs, that is partially or completely complementary to the cleavage site of the enzyme E1_{C}; said zone 1 can optionally include one or more mismatches with the sequence of said cleavage site of the enzyme E1_{C}. Said zone 1, which is different from the zone 1 of the adapter AA′, is chosen so as: (i) to bind only the end generated by the enzyme E1_{C} but not that generated by the enzyme E1_{A}, and (ii) not to reconstitute the sequence of the first N-x bases or base pairs of the recognition site of the restriction enzyme E2, by ligation of said adapter CC′ to at least one of the ends of said DNA fragments obtained in a).

According to yet another advantageous embodiment of the method according to the invention, the adapter AA′ as defined in step b) or the adapter CC′ as defined above, comprises, upstream of the zone 1, a zone 2 of at least 6 base pairs that makes it possible to improve the hybridization by extension of the adapter. The sequence of this zone 2 is selected by any appropriate means known in itself, in particular using programs for predicting appropriate sequences that make it possible to optimize the length, the structure and the composition of oligonucleotides (GC percentage, absence of the secondary structures and/or of self-pairing, etc.); preferably, said adapter comprises at least one base located between the zone 1 and the zone 2, different from that which, in the cleavage site of the restriction enzyme E1, is immediately adjacent to the preceding complementary sequence; this base makes it possible not to reconstitute said restriction site after the ligation of the adapter in step b) and therefore to prevent cleavage of the adapter linked to the end of said double-stranded DNA fragment.

According to yet another advantageous embodiment of the method according to the invention, the adapter AA′ as defined in step b) and/or the adapter CC′ as defined above comprise a phosphate residue covalently linked to the 5′ end of the strand A′ and/or C; this phosphate residue enables an enzyme such as T4 DNA ligase to link said adapter to the 3′-OH ends of the double-stranded DNA fragment (F1), by means of a phosphodiester bond.

According to yet another advantageous embodiment of the method according to the invention, the adapter AA′ as defined in step b) and/or the adapter CC′ as defined above are linked, at the 5′ end of the strand A and/or C′, to different labels.

According to an advantageous arrangement of this embodiment, the 5′ end of the strand C′ of the adapter CC′ is linked to a label that can attach to a functionalized solid support.

The functionalized solid supports that make it possible to attach nucleic acids are known to those skilled in the art. By way of nonlimiting example, mention may in particular be made of magnetic beads functionalized with streptavidin (binding to a biotin-labeled nucleic acid molecule), or an anti-fluoresceine or anti-digoxigenin antibody (binding to a nucleic acid molecule labeled with fluoresceine or digoxigenin), or alternatively other functionalized supports such as nonmagnetic beads made of a polymer or a gold surface, that are functionalized.

According to another advantageous arrangement of this embodiment, the adapter AA′ is linked to a label for detecting nucleic acid hybrids (DNA-DNA or DNA-RNA), for example a fluorophore.

According to yet another advantageous embodiment of the method according to the invention, when said method comprises a single selection of short fragments according to steps a) to d) as defined above, it comprises at least one additional step b′), c′) and/or d′), respectively between steps b) and c) or c) and d), or else after step d), consisting of the amplification of the fragments F′1 or F2 using an appropriate pair of primers, preferably a pair of primers labeled with a label as defined above.

Preferably, the fragments F′1 are amplified using a pair of primers AA′ or AC′ in which the sequence of the primers A, A′ and C′ is that of one of the strands of the adapters AA′ and CC′ as defined above, the primer A and/or the primer C′ being optionally linked, in the 5′ position, respectively with a label for detecting nucleic acid hybrids (DNA-DNA or DNA-RNA) and a label that can attach to a functionalized solid support, as defined above. Preferably, the short fragments F2 are linked, in the 3′ position, with a mixture of adapters complementary to all the 3′ ends of said fragments F2 that can be generated by said restriction enzyme E2, and then said short fragments F2 are amplified using a (sense) primer A as defined above, preferably linked, in the 5′ position, to a label for detecting nucleic acid hybrids (DNA-DNA or DNA-RNA), and a mixture of antisense primers corresponding to the mixture of the sequences of one of the strands of the above adapter mixture.

According to yet another advantageous embodiment of the method according to the invention, when steps a) and b) are carried out, respectively, with two different restriction enzymes E1_{A} and E1_{C} and two different adapters AA′ and CC′ such that the adapter AA′ or CC′ is linked to a label that can attach to a functionalized solid support, the fragments F′1 obtained in step b) or b′) are brought into contact with said functionalized support prior to the cleavage step c), and the fraction of short fragments F2 of step d) corresponds to the fraction of fragments that is either retained on said support (adapter AA′ linked to the label that attaches to the support) or free (adapter CC′ linked to the label that attaches to the support).

Said free fraction is recovered by any means known to those skilled in the art, in particular by centrifugation or magnetization of the functionalized support (beads).

Said fraction retained on the support can optionally be recovered by denaturation of the double-stranded DNA, in particular with sodium hydroxide, or else by amplification using a pair of appropriate primers, in particular with a sense primer A and a mixture of antisense primers as defined above.

In accordance with the method of the invention, said short fragments F2 obtained in step d) comprise one end consisting of the adapter AA′, and the other end (free end), which is preferably cohesive, comprises a random sequence of a few bases (less than 10), generated by cleavage with the restriction enzyme E2 (FIG. 2); consequently, it is possible to select one or more subsets of short fragments F′2 by ligation with an adapter (BB′) or several different adapters (B₁B₁′, B₂B₂′, etc.), each comprising, at the 5′ end of the strand B or at the 3′ end of the strand B′, a specific cohesive sequence of 1 to 10 bases, complementary to the 3′ end of a subset of short fragments F′2 (FIG. 3). Said subset(s) of fragments F′2 is (are) amplified, independently or simultaneously, by PCR using a pair of primers whose sequence is complementary to that of the strands A and B′ of the adapters as defined above.

In accordance with the method of the invention, the adapter BB′ as defined in step e) is an oligonucleotide of at least 6 bp, made up of two complementary strands (B and B′), selected by any appropriate means known in itself, in particular using programs for predicting appropriate sequences that make it possible to optimize the length, the structure and the composition of oligonucleotides (GC percentage, absence of secondary structures and/or of self-pairing, etc.).

In accordance with the method of the invention, said adapter in e) is linked to the ends of said short fragments F2 by any appropriate means known in itself, in particular using a DNA ligase such as T4 ligase.

In accordance with the method of the invention, the amplification in step f) is carried out in particular by PCR using a pair of primers whose sense and antisense sequences are, respectively, those of the strand A and of the strand B′ of the adapters as defined above.

According to an advantageous arrangement of this embodiment, step e) comprises the ligation—simultaneously or independently, preferably independently—of one end of the short fragments F2 obtained in d) to several different adapters (B₁B₁′, B₂B₂′, etc.), each comprising—at the 5′ end of the strand B or at the 3′ end of the strand B′—a specific sequence of 1 to 10 bases, complementary to the free 3′ end of said short fragment F2. Such an arrangement advantageously makes it possible to obtain subgroups of fragments F′2, each corresponding to a different genetic footprint or marker; thus, adapters having specific sequences of n bases make it possible to obtain 4ⁿ subgroups of different genetic footprints (FIG. 2).

According to another advantageous arrangement of this embodiment, said adapter BB′ (step e) comprises a phosphate residue covalently linked to the 5′ end of the strand B; this phosphate residue enables an enzyme such as T4 DNA ligase to link said adapter to the 3′-OH end of the short fragment F2 by means of a phosphodiester bond.

According to yet another advantageous arrangement of this embodiment, one of the primers (step f) is linked, at its 5′ end, to an appropriate label for detecting nucleic acid hybrids (DNA-DNA or DNA-RNA), for example a fluorophore.

According to yet another advantageous arrangement of the above embodiments, they comprise an additional step d″) or g) consisting of the obtaining, by any appropriate means, of single-stranded fragments from the short fragments F2 obtained in step d) or d′) or else from the short fragments F′2 obtained in step f). Preferably, one of the strands of the short fragment obtained in step d), d′) or f) is protected at its 5′ end with an appropriate label; such a label makes it possible in particular to eliminate the complementary strand through the action of phosphatase and then 5′-exonuclease.

According to yet another advantageous arrangement of the above embodiments, they comprise an additional step consisting of the purification, by any appropriate means, of the amplification products obtained in step b′), c′), d′) or f) or of the single-stranded fragments obtained in steps d″) or g).

A subject of the present invention is also a DNA fragment, representing a genetic marker, that can be obtained by means of the method as defined above, characterized in that it has a sequence of less than 100 bases or base pairs, comprising at least one specific sequence consisting of a fragment of genomic DNA or of cDNA bordered, respectively, by the recognition site and the cleavage site of a restriction enzyme E2, the cleavage site of which is located downstream of said recognition site, such that the 5′ end of said specific sequence corresponds to the last x base pairs of the recognition site—having N base pairs—of said enzyme E2, with 1≦x≦N−1, said marker including, at each end, at least 6 bases or 6 base pairs of nonspecific sequence.

According to an advantageous embodiment of said DNA fragment, it is a single-stranded fragment.

According to another advantageous embodiment of said DNA fragment, it is linked, at one of its 5′ ends, to an appropriate label for detecting nucleic acid hybrids (DNA-DNA or RNA-DNA), for example a fluorophore.

A subject of the present invention is also an appropriate support, in particular a miniaturized support of the DNA chip type, comprising said DNA fragment. The supports on which nucleic acids can be immobilized are known in themselves; by way of nonlimiting example, mention may be made of those that are made of the following materials: plastic, nylon, glass, gel (agarose, acrylamide, etc.) and silicon.

Besides the DNA fragments as defined above, a subject of the invention is also the mixtures of DNA fragments corresponding to the subsets of short fragments F′2 obtained in step f) or g); said fragments or mixtures thereof as defined above are useful as genetic markers for analyzing genomes and transcriptomes, in particular for detecting a species, a variety or an individual (animal, plant, microorganism), detecting a gene polymorphism, and for establishing gene expression profiles.

Consequently, a subject of the present invention is also the use of a DNA fragment as defined above, or else of mixtures of DNA fragments corresponding to the subsets of short fragments F′2 obtained in step f) or g) of the method as defined above, as genetic markers.

A subject of the present invention is also a method of hybridizing nucleic acids, characterized in that it uses a DNA fragment as defined above.

A subject of the present invention is also a kit for carrying out a method of hybridization, characterized in that it comprises at least one DNA fragment (target or probe) as defined above; preferably, when said fragment is a target, said kit also comprises a nucleic acid molecule complementary to said DNA fragment, in particular an oligonucleotide probe.

A subject of the present invention is also the use of at least one adapter AA′ as defined above, in combination with an enzyme E2 as defined above, for preparing DNA fragments as defined above.

A subject of the present invention is also a kit for carrying out the method as defined above, characterized in that it comprises at least one adapter AA′ and an enzyme E2 as defined above; preferably, said kit also comprises at least one adapter BB′ and a pair of primers as defined above.

Besides the above arrangements, the invention also comprises other arrangements that will emerge from the following description, which refers to examples of embodiment of the method according to the invention and of its use for analyzing genomes by hybridization with oligonucleotide probes immobilized on a miniaturized support of the DNA chip type, and also to the attached drawings in which:

FIG. 1 illustrates steps a) to d) of the method according to the invention; to simplify the figure, only the strand A of the adapter AA′ is annotated;

FIG. 2 illustrates, by means of examples of cleavage sites of the restriction enzyme E1 and of recognition sites of the restriction enzyme E2, the fraction of short fragments F2 that is selected in step d) and the number of potential subgroups of footprints obtained in step f), deduced from the number of bases selected, respectively, at the 5′ (step b) and 3′ (step e) ends of said short DNA fragments;

FIG. 3 illustrates steps e) and f) of the method according to the invention; to simplify the figure, only the strands A and B of the adapters AA′ and BB′ are annotated;

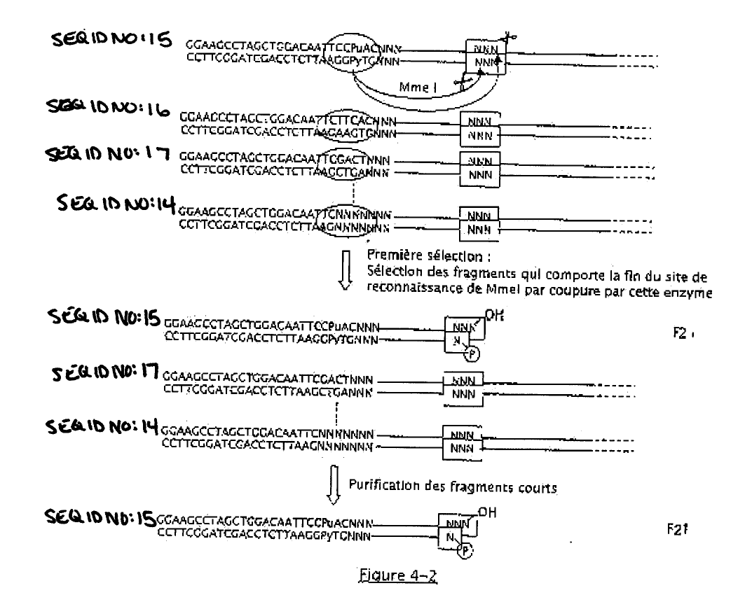

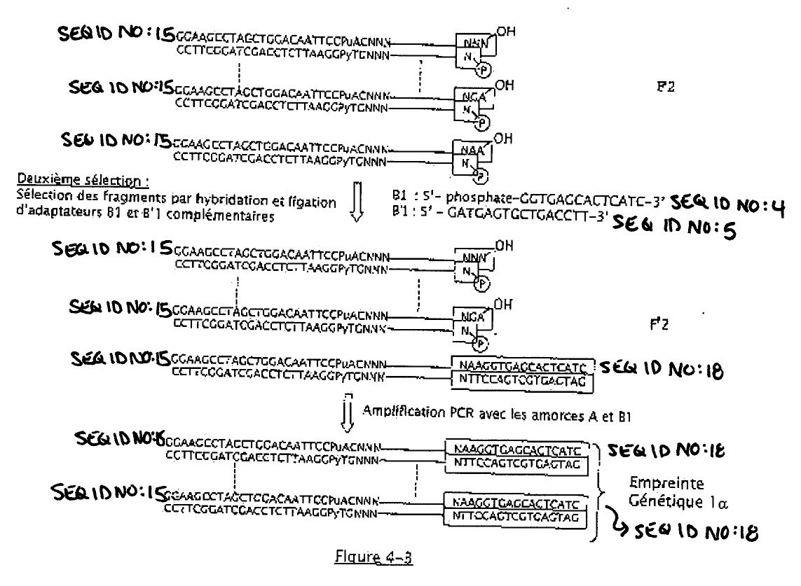

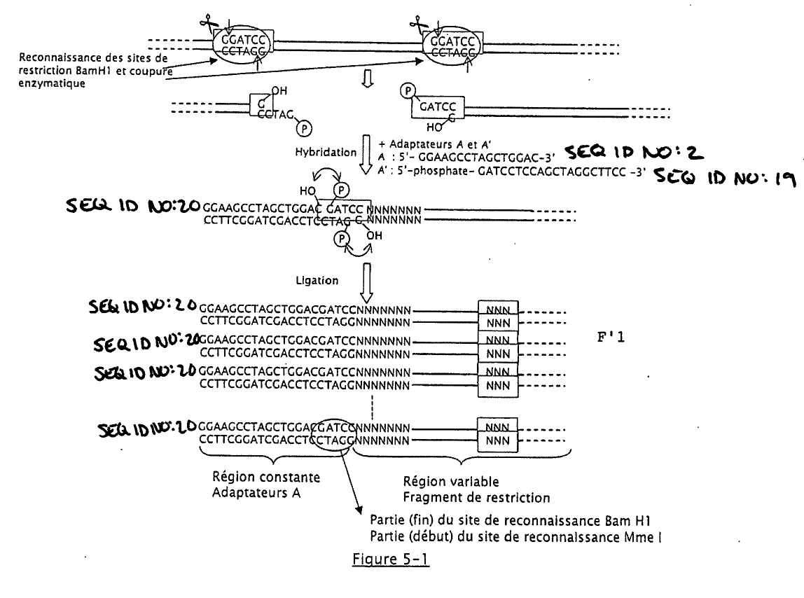

FIGS. 4-1 to 4-3 illustrate a first example of steps a) to f) of the method according to the invention; FIG. 4-1: steps a and b, FIG. 4-2: steps c and d, FIG. 4-3: steps e and f;

step a): the double-stranded DNA fragments are generated by cleavage with EcoR I, which recognizes the GAATTC site,

step b): the adapter AA′ (16/20 bp) comprises, respectively from 5′ to 3′: 15 base pairs (zone 2: GGAAGCCTAGCTGGA (SEQ ID NO:1) on the strand A) and 1 base pair not complementary to the EcoR I site (C on the strand A), and also 4 bases complementary to the EcoR I site (zone 1) including the 5′ end of the Mme I site (A) and a phosphate residue, at the 5′ end of the strand A′ (5′phosphate-AATT). DNA ligase makes it possible to link the adapter to the cohesive ends of the EcoR I fragments by means of phosphodiester bonds, and

steps c) and d): the fragments linked to the adapter are cleaved with the Mme I enzyme (enzyme E2) so as to generate short fragments (45/43 bp) from the fragments that have restored the Mme I site (TCCPuAC) by ligation of the adapter AA′ with a fragment whose 5′ end corresponds to the sequence CPuAC; the selection of 4 specific base pairs (CPuAC) makes it possible to decrease the number of fragments by a factor of 128 (4×2×4×4), compared with the starting sample,

step d): the short fragments obtained in step c) are purified,

step e): the short fragments purified in step d) are linked, at one of their ends, to an adapter B₁B₁′ (14/16 bp) comprising 2 bases complementary to the end of said fragment (TT) at the 3′ end of the strand B′₁, and a phosphate group at the 5′ end of the strand B₁; the selection of two specific bases (AA) makes it possible to decrease the number of fragments by a factor of 2048 (4×2×4×4×16) compared with the starting sample and to obtain short fragments of 59 base pairs comprising 28 base pairs specific for the DNA to be analyzed, which fragments correspond to 16 potential subgroups of genetic footprints,

step f): the fragments selected in step e) are amplified using sense and antisense primers corresponding to the sequences complementary, respectively, to the strands A and B′₁ of the adapters AA′ and B₁B′₁;

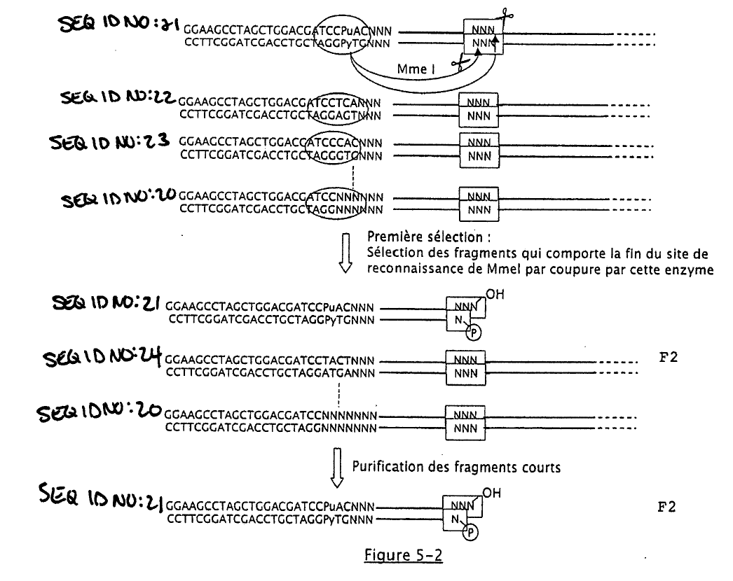

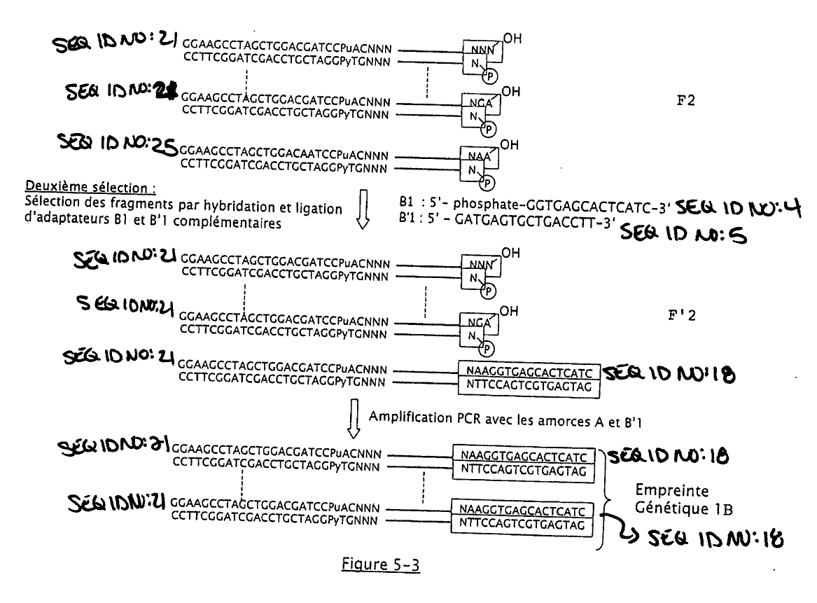

FIGS. 5-1 to 5-3 illustrate a second example of steps a) to f) of the method according to the invention: FIG. 5-1: steps a and b, FIG. 5-2: steps c and d, FIG. 5-3: steps e and f;

step a): the double-stranded DNA fragments are generated by cleavage with BamH I, which recognizes the GGATCC site,

step b): the adapter AA′ (16/20 bp) comprises, respectively from 5′ to 3′: 15 base pairs (zone 2: GGAAGCCTAGCTGGA (SEQ ID NO:1) on the strand A) and 1 base pair not complementary to the BamH I site (C on the strand A), and also 4 bases complementary to the BamH I site (zone 1) including 2 bases of the 5′ end of the Mme I site (AG) and a phosphate residue, at the 5′ end of the strand A′ (5′ phosphate-GATC). DNA ligase makes it possible to link the adapter to the cohesive ends of the BamH I fragments by means of phosphodiester bonds, and

steps c) and d): the fragments linked to the adapter are cleaved with the Mme I enzyme (enzyme 2) so as to generate short fragments (44/42 bp) from the fragments that have restored the Mme I site (TCCPuAC) by ligation of the adapter AA′ with a fragment whose 5′ end corresponds to the sequence PuAC; the selection of 3 specific base pairs (PuAC) makes it possible to decrease the number of fragments by a factor of 32 (2×4×4) compared with the starting sample,

step d): the short fragments obtained in step c) are purified,

step e): the short fragments purified in step d) are linked, at one of their ends, to an adapter B₁B₁′ (14/16 bp) comprising 2 bases complementary to the end of said fragment (TT) at the 3′ end of the strand B′₁, and a phosphate group at the 5′ end of the strand B₁; the selection of 2 specific bases (AA) makes it possible to decrease the number of fragments by a factor of 512 (2×4×4×16) compared with the starting sample and to obtain short fragments of 58 base pairs comprising 28 base pairs specific for the DNA to be analyzed, which fragments correspond to 16 potential subgroups of genetic footprints,

step f): the fragments selected in step e) are amplified using sense and antisense primers corresponding to the sequences complementary, respectively, to the strands A and B′₁ of the adapters AA′ and B₁B′₁;

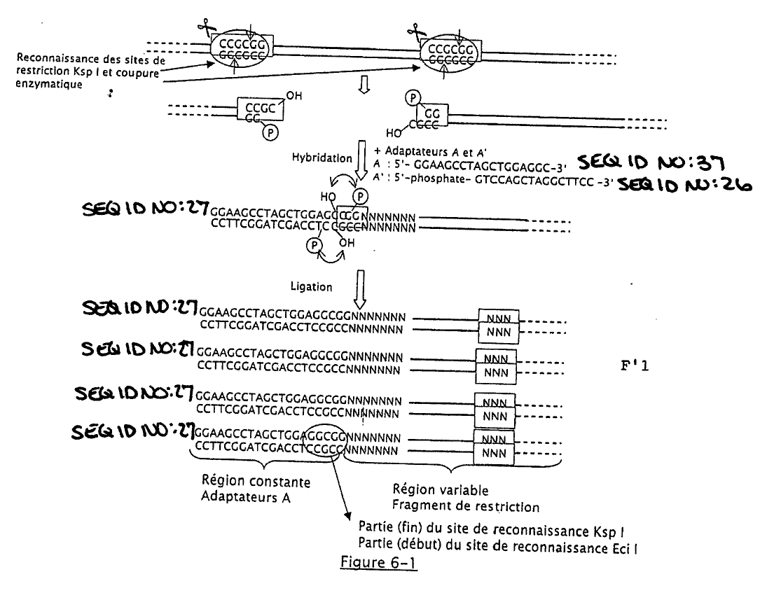

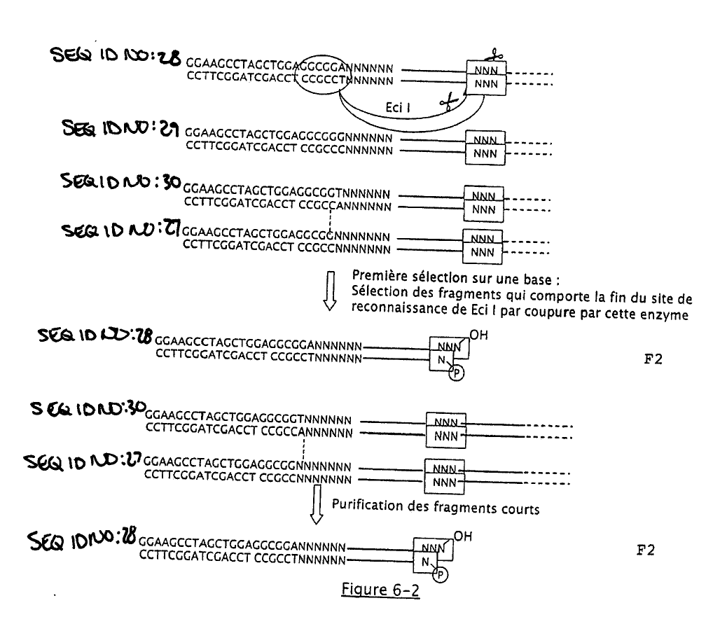

FIGS. 6-1 to 6-3 illustrate a third example of steps a) to f) of the method according to the invention: FIG. 6-1: steps a and b, FIG. 6-2: steps c and d, FIG. 6-3: steps e and f;

step a): the double-stranded DNA fragments are generated by cleavage with Ksp I, which recognizes the CCGCGG site,

step b): the adapter AA′ (18/16 bp) comprises, respectively from 5′ to 3′: 15 base pairs (zone 2: GGAAGCCTAGCTGGA (SEQ ID No. 1) on the strand A), and also one base pair of the 5′ sequence of the Eci I site (G on the strand A) and 2 bases complementary to the Ksp I site (GC on the strand A) and a phosphate residue, at the 5′ end of the strand A′; said adapter including 3 bases of the 5′ end of the Eci I site (GGC). DNA ligase makes it possible to link the adapter to the cohesive ends of the Ksp I fragments by means of phosphodiester bonds, and

steps c) and d): the fragments linked to the adapter are cleaved with the Eci I enzyme (enzyme 2) so as to generate short fragments from the fragments that have restored the Eci I site (GGCGGA) by ligation of the adapter AA′ with a fragment whose 5′ end corresponds to the sequence A; the selection of a specific base pair makes it possible to decrease the number of fragments by a factor of 4 compared with the starting sample,

step d): the short fragments obtained in step c) are purified,

step e): the short fragments purified in step d) are linked, at one of their ends, to an adapter B₁B₁′ (14/16 bp) comprising 2 bases complementary to the end of said fragment (TT) at the 3′ end of the strand B′₁, and a phosphate group at the 5′ end of the strand B1; the selection of 2 specific bases (AA) makes it possible to decrease the number of fragments by a factor of 64 (4×16) compared with the starting sample and to obtain short fragments comprising 28 base pairs specific for the DNA to be analyzed, which fragments correspond to 16 potential subgroups of genetic footprints,

step f): the fragments selected in step e) are amplified using sense and antisense primers corresponding to the sequences complementary, respectively, to the strands A and B′₁ of the adapters AA′ and B₁B′₁;

FIG. 7 illustrates an example of implementation of the method according to the invention using two different enzymes E1 (E1_{A} cleaves frequently, such as Msp I and Taq^{α} I, and E1_{C} cleaves rarely, such as Pst I, EcoR I), so as to further reduce the complexity of the DNA to be analyzed, by introduction of an additional selection through cleavage with the enzyme E1_{C}. The sequences of the restriction sites are indicated in the 5′→3′ direction for the positive strand. The bases indicated in bold remain on the fragment of interest after cleavage. The bases underlined are those that are imposed by the coupling of the E1_{A} and E2 enzymes;

FIG. 8 illustrates an example of implementation of the method according to the invention using two different enzymes E1, so as to select a first set of fragments A=C and then a fraction of short fragments F2 (step c).

### EXAMPLE 1

### Preparation of Short DNA Fragments (Target or Probe) According to the Method of the Invention

The preparation of the nucleic acids, the enzymatic digestions, the ligations, the PCR amplifications and the purification of the fragments thus obtained were carried out using conventional techniques, according to standard protocols such as those described in Current Protocols in Molecular Biology (Frederick M. AUSUBEL, 2000, Wiley and Son Inc. Library of Congress, USA).

The genomic DNA was extracted from bovine blood using the PAXgene Blood DNA kit (reference 761133, QIAGEN), according to the supplier's instructions.

The following adapters and primers were synthesized by MWG Biotech:

| | |
|---|---|
| Adapter AA′ | |
| strand A: | |
| 5′-CGAAGCCTAGCTGGAC-3′ | (SEQ ID No. 2) |
| strand A′: | |
| 5′-P-AATTCTCCAGCTAGGCTTCC-3′ | (SEQ ID No. 3) |
| Adapter BB′ | |
| B: | |
| 5′-P-GGTGAGCACTCATC-3′ | (SEQ ID No. 4) |
| B′: | |
| 5′-GATGAGTGCTGACCTT-3′ | (SEQ ID No. 5) |

Primers

The pair of primers 1:

| | |
|---|---|
| Sense: | |
| 5′-CCTTCGGATCGACCTG-3′ | (SEQ ID No. 6) |
| Antisense: | |
| 5′-CTACTCACGAGTGGAA-3′ | (SEQ ID No. 7) |

or the pair of primers 2:

| | |
|---|---|
| Sense: | |
| 5′-GGAAGCCTAGCTGGAC-3′ | (SEQ ID No. 2) |
| Antisense: | |
| 5′-GATGAGTGCTGACCTT-3′ | (SEQ ID No. 5) |

can be used without distinction.

The pair of primers 2 makes it possible in particular to re-use part of the sequences of the adapters.

The short DNA fragments (F2 and F′2) were then prepared according to the following steps:

1) Digestion of the Genomic DNA with Eco RI and Ligation of the Fragments to the Adapter AA′ (Steps a and b)

The purified genomic DNA (5 μg) and the adapter (5 μg) were incubated at 37° C. for 3 h in 40 μl of 10 mM Tris-HCl buffer, pH 7.5, containing 10 mM MgCl₂, 50 mM NaCl, 10 mM DTT, 1 mM EDTA, 1 mM ATP and 1 mg BSA and containing 50 IU of EcoR I and 2 IU of T4 DNA ligase. The DNA fragments linked at their ends to the adapter AA′ thus obtained were purified by precipitation from a 1:4 (V/V) mixture of 3M ammonium acetate and ethanol.

2) Digestion of the Fragments with Mme I and Selection of the Short Fragments F2 (steps c) and d))

The pellet was resuspended in 40 μl of buffer containing 50 mM potassium acetate, 20 mM Tris-acetate, 10 mM magnesium acetate and 1 mM DTT, pH 7.9, and incubated at 37° C. for 1 h in the presence of 5 IU of Mme I.

3) Purification of the Fraction of Short Fragments F2 (Step d)

The enzyme was removed using the Micropure-EZ kit (Millipore), the salts were subsequently removed by filtration (Microcon YM3), then the DNA retained on the YM3 filter was eluted and the short fragments were purified by filtration (Microcon YM 30 or YM 50, Millipore), the DNA fragments of less than 100 bp corresponding to the eluate, the larger fragments being retained on the filter.

4) Ligation of the Short Fragments F2 to the Adapter BB′ (Step e)

The short fragments obtained in step d) and the adapter BB′ (3 μg) were incubated at 37° C. for 3 h in 40 μl of 10 mM Tris-HCl buffer, pH 7.5, containing 10 mM MgCl₂, 50 mM NaCl, 10 mM DTT, 1 mM EDTA, 1 mM ATP and 1 mg BSA, and containing 2 IU of T4 DNA ligase.

5) Amplification of the Short Fragments Linked to the Adapter BB′ (F′2) (Step f)

The short fragments linked to the adapter BB′, obtained in step e), were subsequently amplified by PCR using the sense and antisense pair corresponding to the sequences complementary to the strands A and B′ of the adapters AA′ and BB′, in a reaction volume of 50 μl containing: 1 ng of DNA fragments, 150 ng of each of the primers and 2 IU of AmpliTaq GOLD® (Perkin Elmer) in a 15 mM Tris-HCl buffer, pH 8.0, containing 10 mM KCl, 5 mM MgCl₂ and 200 μM dNTPs. The amplification was carried out in a thermocycler, for 35 cycles comprising: a denaturation step at 94° C. for 30 s, followed by a hybridization step at 60° C. for 30 s and an extension step at 72° C. for 2 min. The PCR-amplified fragments were purified using the MinElute PCR Purification kit (reference LSKG, Qiagen), according to the supplier's instructions.

The enzyme, the salts and the free dNTPs were removed by filtration on Micropure-EZ (Millipore) and then on Microcon YM3 (Millipore), and the PCR amplification product retained on the filter was then eluted.

### EXAMPLE 2

### Use of the Target DNAs for Hybridizing Oligonucleotide Probes

The short double-stranded DNA fragments (target DNAs) obtained in example 1 were converted to single-stranded DNA by digestion at 37° C. for 30 min in a reaction volume of 40 μl containing 3×10⁻³ IU of 5′-exonuclease in a 0.02 M ammonium citrate buffer, pH 5. The enzyme, the salts and the free dNTPs were removed by filtration on Micropure-EZ (Millipore) and then on Microcon YM3 (Millipore) and the single-stranded DNA retained on the filter was then eluted.

A glass support of the DNA chip type, on which are immobilized oligonucleotide probes, some of which are complementary to the target DNA fragments obtained in example 1, was prepared according to techniques known in themselves. Said target DNAs were then diluted in hybridization buffer (H7140, Sigma) and 10 μl were deposited onto the glass support, between slide and cover slip. The hybridization was then carried out, in a humid chamber in a thermocycler, under the following conditions: 80° C. for 3 min, and then the temperature is lowered to 50° C. in steps of 0.1° C./s and, finally, the temperature is maintained at 50° C. for 10 minutes. The hybridization reaction was then stopped by placing the glass slides on ice.

The excess of target DNA fragments not complementary to the probes was then removed by successive washing: 30 s with 2×SSC (Sigma, S6639), 30 s with 2×SSC to which 0.1% SDS (L4522, Sigma) has been added, and 30 s with 0.2×SSC, at +4° C.

The glass slides were then dried and the hybridization was visualized and analyzed using a scanner (Gentaq model, Genomic Solution).

### EXAMPLE 3

### Preparation of Short DNA Fragments Using Two Different E1 Enzymes, E1_{A} and E1_{C}

The genomic DNA is prepared as described in example 1.

The following adapters and primers were synthesized:

Adapter AA′ (complementary to the Taq^{α} I site)

| | |
|---|---|
| strand A: | |
| 5′-GACGATGAGTCCTGAC-3′ | (SEQ ID No. 8) |
| strand A′: | |
| 5′-P-CGGTCAGGACTCATCGTC-3′ | (SEQ ID No. 9) |

Adapter CC′ (complementary to the EcoR I site)

| | |
|---|---|
| strand C: | |
| 5′P-AATTGGTACGCAGTCTAC-3′ | (SEQ ID No. 10) |
| strand C′: | |
| 5′-GTAGACTGCGTACC-3′ | (SEQ ID No. 11) |
| Primers | |
| sense primer: | |
| 5′-Cy3-GACGATGAGTCCTGACCG-3′ | (SEQ ID No. 12) |
| antisense primer: | |
| 5′-biotin-GTAGACTGCGTACCAATT-3′. | (SEQ ID No. 13) |

The short DNA fragments (F2) were then prepared according to the following steps:

1) Digestion of the Genomic DNA with Eco RI and Tag^{α} I and Ligation of the Fragments to the Adapters AA′ and CC′ (Steps a and b)

The purified genomic DNA (5 μg) and each of the adapters (5 μg of AA′ and 5 μg of CC′) were incubated at 37° C. for 3 h in 40 μl of 10 mM Tris-HCl buffer, pH 7.5, containing 10 mM MgCl₂, 50 mM NaCl, 10 mM DTT, 1 mM EDTA, 1 mM ATP and 1 mg BSA, and containing 50 IU of EcoR I, 50 IU of Taq^{α} I and 2 IU of T4 DNA ligase.

2) Amplification of the fragments F′1

The DNA fragments F′1 linked in the 5′ position to the adapter AA′ and in the 3′ position to the adapter CC′ were amplified using the sense and antisense primers (SEQ ID Nos. 8 and 11) in a reaction mixture with a final volume of 50 μl containing 1 μl of ligated fragments, 2 IU of polymerase (AmpliTaq Gold, Perkin-Elmer), 150 ng of each of the primers and 200 μM of each of the dNTPs in a Tris-HCl buffer, pH 8, containing 10 mM KCl and 5 mM MgCl₂. The amplification was carried out under the following conditions: 35 cycles comprising a denaturation step at 94° C. for 30 s, a hybridization step at 60° C. for 30 s, and then an elongation step at 72° C. for 2 min.

3) Binding of the Fragments F′1 (A=C) to Functionalized Magnetic Beads

Resuspended magnetic beads functionalized with streptavidin (Dynal or Molecular Probes; 500 μg) are placed in the vicinity of a magnet so as to form a pellet, and the supernatant is then removed. The beads are rinsed twice in 50 μl of buffer (2 M NaCl, 10 mM Tris-HCl, pH 7.5, 1 mM EDTA) and then resuspended in 100 μl of buffer (1 M NaCl, 5 mM Tris-HCl, pH 7.5, 0.5 mM EDTA). The PCR reaction product (50 μl) is added to the suspension of beads, and the mixture is then vigorously agitated and then incubated, with agitation, at ambient temperature for 30 min. The supernatant is then removed by magnetization as above, and the beads are rinsed with 100 μl of 0.1×SSC buffer containing 0.1% SDS.

3) Digestion of the Fragments F′1 (A=C) with BseR I and Purification of the Short Fragments F2 (Steps c and d)

The pellet formed by the beads was resuspended in 40 μl of BseR I enzyme reaction buffer and incubated at 37° C. for 3 h in the presence of 5 IU of BseR I. The short fragments F2 that have been released into the reaction medium are recovered.

4) Target DNA Hybridization

A glass support of the DNA chip type, on which are immobilized oligonucleotide probes, some of which are complementary to the target DNA fragments obtained, was prepared according to techniques known in themselves. Said target DNAs were then denatured at 95° C. for 3 min and diluted in hybridization buffer (H7140, Sigma) and 10 μl were deposited onto the glass support, between slide and cover slip. The hybridization was then carried out, in a humid chamber, for 2 hours at 50° C. The hybridization reaction was then stopped by placing the glass slides on ice.

The excess of target DNA fragments not complementary to the probes were then removed by successive washing: 30 s with 2×SSC (Sigma, S6639), 30 s with 2×SSC to which 0.1% SDS (L4522, Sigma) has been added, and 30 s with 0.2×SSC, at +4° C.

The glass slides were then dried and the hybridization was visualized and analyzed using a scanner (Gentaq model, Genomic Solution).

As emerges from the above, the invention is in no way limited to those of its methods of implementation, execution and application which have just been described more explicitly; on the contrary, it encompasses all the variants thereof that may occur to those skilled in the art, without departing from the context or the scope of the present invention.

1. A method of preparing DNA fragments from a sample of nucleic acids to be analyzed, which method comprises selectively fragmenting the nucleic acids by means of at least the following steps:
I. for a first selection of short fragments:
a) preparing first double-stranded DNA fragments F1 using at least one restriction enzyme E1 capable of randomly fragmenting the sample of nucleic acids to be analyzed, generating said DNA fragments F1 with blunt or cohesive ends,
b) ligating the ends of said DNA fragments F1 obtained in step a) to at least one adapter AA′, so as to form a unit—located at the junction of the complementary end of said adapter and of the 5′ end of said fragments F1, such that:
the sequence of said unit is that of the first N-x base pairs of the recognition site—comprising N base pairs—of a restriction enzyme E2, the cleavage site of which is located downstream of said recognition site, with 1≦x≦N−1, and
its 3′ end—located 5′ of said DNA fragments F1—is that of the restriction site of the E1 restriction enzyme, so as to obtain DNA fragments F′1,
c) cleaving the DNA fragments F′1 obtained in b) in the vicinity of their 5′ end using said restriction enzyme E2, so as to select a fraction of short fragments F2,
d) purifying said fraction of short fragments F2, and, optionally,
II. for a second selection of one or more subset(s) of fragments from the fraction of short fragments F2 obtained in step d):
e) litgating the free end (not linked to the adapter AA′) of short fragments F2 obtained in d) to at least a second complementary adapter BB′ (production of fragments F′2), and
f) amplifying the short fragments F′2 linked to said adapters (AA′ and BB′), using at least one pair of appropriate primers, at least one being optionally labeled at its 5′ end, so as to select at least one subset of short fragments F′2 from the fraction of short fragments F′2 obtained in d).

2. The method as claimed in claim 1, wherein step a) is carried out with two different E1 restriction enzymes, E1_{A} and E1_{C}, such that:
at least one generates cohesive ends, different from those optionally generated by the other restriction enzyme, and
the 3′ end of the E1_{1A} restriction site is that of the unit as defined in step b).

3. The method as claimed in claim 2, wherein one of the enzymes cleaves frequently and the other rarely.

4. The method as claimed in claim 3, wherein:
the enzyme that cleaves frequently is the enzyme E1_{A}, which enzyme E1_{A} generates at least one end of a fragment F1 that binds to the adapter AA′ in step b), and
the enzyme E1_{C} that cleaves rarely, generates at least one end of a fragment F1, which binds, in step b), to a second adapter CC′ that is different from the adapter AA′.

5. The method as claimed in claim 1, wherein steps a) and b) are carried out simultaneously.

6. The method as claimed in claim 1, which further comprises purifying the fragments less than 1000 bp, prior to the ligation step b).

7. The method as claimed in claim 1, wherein the adapter AA′ as defined in step b) comprises, at the 3′ end of the strand A or 5′ end of the strand A′, or both, a zone 1 of approximately 1 to 8 bases or base pairs, which is partially or completely identical or complementary to the restriction site of the enzyme E1, which zone 1 is chosen so as to reconstitute the sequence of the first N-x bases or base pairs of the recognition site of the restriction enzyme E2, by ligation of said adapter AA′ to the ends of said DNA fragments obtained in a).

8. The method as claimed in claim 7, wherein zone 1 includes one or more mismatches with the sequence of said cleavage site of the restriction enzyme E1.

9. The method as claimed in claim 1, wherein the adapter as defined in step b) comprises, upstream of the zone 1, a zone 2 of at least 6 base pairs.

10. The method as claimed in claim 1, wherein the adapter as defined in step b) comprises at least one base located between the zone 1 and the zone 2, different from that which, in the cleavage site of the restriction enzyme E1, is immediately adjacent to the complementary sequence corresponding to the zone 1.

11. The method as claimed in claim 1, wherein the adapter as defined in step b) comprises a phosphate residue covalently linked to the 5′ end of the strand A′.

12. The method as claimed in claim 1, wherein, when said method consists of a single selection of short fragments according to steps a) to d), it comprises at least one additional step b′), c′) or d′) or a combination thereof comprising amplifying the fragments F′1 or F2 using an appropriate pair of primers, preferably a pair of labeled primers.

13. The method as claimed in claim 1, wherein the adapter AA′ as defined in step b) is linked, at the 5′ end of its strand A, to an appropriate label, in particular a label for detecting nucleic acid hybrids or a label that is attachable to a functionalized solid support.

14. The method as claimed in claim 1, wherein the 5′ end of the strand C′ of the adapter CC′ is linked to a label, which label is attachable to a functionalized solid support.

15. The method as claimed in claim 1, wherein the fragments F′1 obtained in step b) or b′) are brought into contact with said functionalized support prior to the cleavage step c), and the fraction of short fragments F2 of step d) corresponds to the fraction of fragments that is either retained on said support (adapter AA′ linked to the label that attaches to the support) or free (adapter CC′ linked to the label that attaches to the support).

16. The method as claimed in claim 13, which comprises, in step e), ligating several different complementary adapters (B₁B₁′, B₂B₂′, etc.), each comprising, at the 5′ end of the strand B or at the 3′ end of the strand B′, a specific sequence of 1 to 10 bases.

17. The method as claimed in claim 13, wherein said adapter BB′ as defined in step e) comprises a phosphate residue covalently linked to the 5′ end of the strand B.

18. The method as claimed in claim 13, wherein one of the primers as defined in step f) is linked, at its 5′ end, to an appropriate label.

19. The method as claimed in claim 1, which comprises an additional step d″) or g) comprising obtaining single-stranded fragments from the short fragments F2 obtained in step d) or d′) or else from the short fragments F′2 obtained in step f).

20. The method as claimed in claim 1, which further comprises purifying the amplification products obtained in step b′), c′), d′) or f) or of the single-stranded fragments obtained in step d″) or g).

21. A short DNA fragment, representing a genetic marker, obtained by the method as claimed in claim 1, which has a sequence of less than 100 bases or base pairs, comprising at least one specific sequence consisting of a fragment of genomic sequence or of cDNA sequence bordered, respectively, by the recognition site and the cleavage site of a restriction enzyme E2, the cleavage site of which is located downstream of said recognition site, such that the 5′ end of said specific sequence corresponds to the last x base pairs of the recognition site—having N base pairs—of said enzyme E2, with 1≦x≦N−1, said marker including, at each end, at least 6 bases or 6 base pairs of nonspecific sequence.

22. The DNA fragment as claimed in claim 21, which is a single-stranded fragment.

23. The DNA fragment as claimed in claim 21 which is linked, at one of its 5′ ends, to an appropriate label.

24. A DNA chip, characterized in that it comprises a DNA fragment as claimed in claim 21.

25. (canceled)

26. (canceled)

27. A method of hybridizing nucleic acids, which comprises hybridizing the nucleic acids with a DNA fragment as claimed in claim 21.

28. A kit for carrying out the method of claim 27.

29. (canceled)

30. (canceled)

31. A kit for carrying out the method as claimed in claim 1, which comprises at least one adapter AA′ as defined in claim 7, and a restriction enzyme E2 as defined in claim 1.

32. The kit as claimed in claim 31, which further comprises at least one adapter BB′ as defined in claim 1, and a pair of primers as defined in claim 1.

33. The kit is claimed in claim 28, which comprises at least one DNA fragment as claimed in claim 21.

34. The kit as claimed in claim 28, which comprises at least one DNA chip as claimed in claim 24.

35. The kit as claimed in claim 33, which further comprises an oligonucleotide probe complimentary to the DNA fragment.

36. A method of hybridizing nucleic acids, which comprises hybridizing the nucleic acids with a DNA chip as claimed in claim 24.
